(19) United States
(12) Patent Application Publication
(10) Pub. No.: US 2020/0390174 A1
(43) Pub. Date: Dec. 17, 2020

(54) MAGNETICALLY ATTACHABLE EYELASH PROSTHETIC SYSTEM AND RELATED METHODS

(71) Applicant: LashLiner, LLC, Woodinville, WA (US)

(72) Inventor: Laura A. Hunter, Mill Creek, WA (US)

(21) Appl. No.: 16/999,384

(22) Filed: Aug. 21, 2020

Related U.S. Application Data
(63) Continuation of application No. 16/287,649, filed on Feb. 27, 2019.
(60) Provisional application No. 62/636,792, filed on Feb. 28, 2018.

(51) Int. Cl. A41G 5/02; A61Q 1/10; A61K 8/81; A61K 8/19

(52) U.S. Cl. CPC A41G 5/02; A61Q 1/10; A61K 8/8152; A61K 2800/412; A61K 2800/47; A61K 2800/413; A61K 8/19

(57) ABSTRACT

Various magnetically eyelash attaching prosthetic systems of the present disclosure are provided, comprising: (a) a load-bearing magnetic eyeliner adherable to an eyelid surface and incorporating magnetic particles compatible with a load-bearing structural adhesive; and (b) a magnetic eyelash prosthetic incorporating one or more magnetic elements exhibiting high magnetic coercivity and positioned at least at the base of the eyelash prosthetic, enabling the interaction between the ferromagnetic particles (incorporated into the load-bearing eyeliner) and the ferromagnetic elements (incorporated into the magnetic eyelash prosthetic) to stably attach the eyelash prosthetic to a pre-coated eyelid surface. Various methods for producing and using the various magnetically attachable eyelash prosthetics and the various load-bearing magnetic eyeliners are also provided.

### CROSS-REFERENCE TO RELATED SPECIFICATION

This application claims the benefit of Provisional Application No. 62/636,792, filed on Feb. 28, 2018, the contents of which are incorporated herein by reference.

### TECHNICAL FIELD

The present disclosure relates to a prosthetic system for magnetically attaching eyelashes to eyelids/eye-contouring skin for cosmetic enhancement. Methods for making and using the disclosed prosthetic system for magnetically and reversibly attaching/detaching one or more eyelash prosthetic units are also provided.

### BACKGROUND OF THE INVENTION

The cosmetic industry is estimated to be one of the fastest growing economic sectors, reaching multi-billion dollars in global annual sales. The global demand for innovative beauty products is incessant, and manufacturers are responding to the demands by developing healthier and more visually pleasing products that can provide multiple benefits in comparison to the lackluster performance of yesterday's products.

In particular, beauty formulations that can be applied to the facial skin are especially in demand. Mascara and eyeliners are particularly desirable by consumers because these beauty-enhancing products provide substantial benefits to their consumers by making a visible impact on their appearances, and thereby, improving one's self-confidence. Mascaras can be formulated to coat native eyelashes with highly pigmented and lash-thickening agents to cosmetically exaggerate the thickness and length of native eyelashes with minimal effort. Women populations are “demanding” more innovative products capable of taking their ordinary-looking eyelashes to a more favorable presentation of lush/thick/lengthy lashes. Product developers are responding by inventing a broad variety of “cosmetic lashes” or “eyelash prosthetics,” which can be manufactured from human hair, animal hair, insect-derived fiber, and various synthetic blends thereof. Because wearing cosmetic eyelashes can dramatically enhance the size/prominence of the user's eyes, the demand for lash prosthetics is on the rise on a global level, among women of a broad age group. However, conventional eyelash prosthetics pose some inconveniences, which may dissuade a majority of women from trying them at all. For many, the daily application of eyelash prosthetics can be tiresome and time-consuming, requiring a steady pair of hands, and over-exposing the delicate eyelid dermis to potentially toxic glue formulations that may be wrinkle-promoting.

To avoid these unpleasant user experiences associated with using sticky glue adhesives, alternative lash-enhancing products have been introduced to the market with mixed reviews. Recently, magnetic lashes containing small magnets have been developed so that can be superimposed against each other as a top/bottom pair, intended to be positioned over and under native eyelashes for their stabilization. These magnetic lashes are not without technical challenges in applying them, often requiring multiple attempts to properly align the top magnetic lash over the bottom magnetic lash in order to stably secure them around the native eyelashes.

There is an unmet need for more advanced and reliable eyelash prosthetic products that are less toxic, more convenient to apply/remove, more durable, and more comfortable without sacrificing the beautiful aesthetics of lush/lengthy prosthetic lashes for cosmetic enhancement.

### SUMMARY

In various embodiments of the present invention, various magnetically eyelash attaching prosthetic systems of the present disclosure are provided, comprising: (a) a load-bearing magnetic eyeliner adherable to an eyelid surface and incorporating magnetic particles compatible with a load-bearing structural adhesive; and (b) a magnetic eyelash prosthetic incorporating one or more magnetic elements exhibiting high magnetic coercivity and positioned at least at the base of the eyelash prosthetic, wherein the adhesive force between the load-bearing structural adhesive of the skin-adhered eyeliner and the eyelid surface exceeds the magnetic force generated between the load-bearing eyeliner and the eyelash prosthetic, wherein the magnetic force between the load-bearing eyeliner and the eyelash prosthetic exceeds the gravitational force acting on the eyelash prosthetic, and thereby, the interaction between the magnetic particles (incorporated into the load-bearing eyeliner) and the magnetic elements (incorporated into the magnetic eyelash prosthetic) stably attaches the eyelash prosthetic to an eyelid surface.

In other embodiments, a load-bearing magnetic eyeliner capable of attaching one or more magnetic eyelash prosthetics is provided, comprising: (a) a pigment suitable for eyelid cosmetic formulation; (b) one or more magnetic particles suitable for use in a pigmented cosmetic formulation; and (c) a load-bearing structural adhesive, capable of adhering to the surface of an eyelid and capable of magnetically supporting the attachment of a magnetic eyelash prosthetic, wherein the cosmetic formulation is convertible from a gel state to a load-bearing adhesive state after coating the skin surface; and wherein the adhesive force between the load-bearing structural adhesive of the skin-adhered eyeliner and the eyelid surface is greater than the magnetic force between the load-bearing eyeliner and the eyelash prosthetic, so that the eyelash prosthetic is reversibly attachable and detachable.

In another embodiment, a magnetically attachable eyelash prosthetic is provided, comprising: (a) a lash base formed in part from a silicone polymer, and incorporating one or more magnetic elements exhibiting high magnetic coercivity; and (b) a plurality of eyelash hairs adhered to the lash base, wherein the magnetic elements are positioned in a portion of the lash base, and arranged to magnetically interact with the surface of a magnetic eyeliner film formable over an eyelid.

In other embodiments, various methods for producing and using the various magnetically attachable eyelash prosthetics and the various load-bearing magnetic eyeliners are provided throughout the specification, including Examples 1-5 providing exemplary formulations for forming the load-bearing magnetic eyeliners and Examples 6-10 providing exemplary formulations for forming the magnetically attachable eyelash prosthetics of the present invention.

### BRIEF DESCRIPTION OF THE DRAWINGS

FIG. 1A illustrates a simplified schematic of a magnetically attachable eyelash prosthetic system from a side perspective, showing the vertical alignment between a load-bearing magnetic eyeliner and a magnetically attachable eyelash prosthetic, as one embodiment.

FIG. 1B illustrates a conceptual schematic of a magnetically attachable eyelash prosthetic system from a top perspective for facilitating the detailed descriptions herein, as one embodiment.

FIG. 2 A-B illustrate a hypothetical pair of eyes without any eyelash adornment, representing the condition prior to the attachment of magnetically attachable eyelash prosthetics of the present invention.

FIG. 3 A-B illustrate the application of the load-bearing magnetic eyeliner to the edge of the eye-lid contour, as one embodiment.

FIG. 4 A-B illustrate the stable attachment of the magnetically attachable eyelash prosthetic to the pre-coated, load-bearing magnetic eyeliner, as one embodiment.

FIG. 5A illustrates the magnetically attachable eyelash prosthetic showing physically attachable magnetic elements, as one embodiment.

FIG. 5B illustrates the magnetically attachable eyelash prosthetic incorporating magnetic elements into the lash base as another embodiment.

FIG. 6A illustrates the magnetically attachable eyelash prosthetic comprising a magnetic lash base formed as a single continuous unit capable of attaching to a load-bearing magnetic eyeliner, as another embodiment.

FIG. 6B illustrates the magnetically attachable eyelash prosthetic comprising a magnetic lash base formed as a dis-continuous unit, as another embodiment.

FIG. 6C illustrates the magnetically attachable eyelash prosthetic formed by attaching several shorter prosthetic units arranged in tandem, as another embodiment.

### DETAILED DESCRIPTION

### A. Definitions

The term “a” refers to one or more of an item/material of interest as typically employed in patent documents.

The term “plurality” refers to one or more of an item/material of interest, interchangeable with the meaning of “many” or “several” or “a set.”

The term “cosmetic” refers to the external application of certain formulations or procedures intended to improve or restore a user's appearance, including the thickening and lengthening appearance of eyelashes for cosmetic enhancement.

The term “pigment” refers to a coloring agent that can be derived from natural or synthetic sources in order to make the product more desirable from a user's perspective. The pigments suitable for making the load-bearing magnetic eyeliner can be selected from a range of materials providing the colors of interest, including black, brown, gray, blue, violet, red, green, orange, yellow and various blends thereof.

The term “dermal compatible” refers to the biocompatibility relative to skin tissue for ensuring the safety of product users for the load-bearing magnetic eyeliner and the magnetic eyelash prosthetics. The terms “cosmetic eyelashes” or “eyelash prosthetics” refer to non-native (manufactured) eyelashes for cosmetic application/adornment/enhancement to improve the user's appearance by thickening and/or lengthening the native eyelashes.

The terms “eyelashes” or “eyelash hairs” are used interchangeably herein to mean “many” or “several” or “a set” of individual hairs derived from human, animals, insects and/or synthetically manufactured blends of natural or man-made materials in any combination or relative ratios.

The term “structural adhesives” refers to an adhesive capable of “hardening” or “curing” into a material capable of holding two or more substrates together in order to bear the forces suitable for the lifetime performance of the product.

The term “incorporating” refers to the inclusion or the addition of a material substance of interest in any manner, not limited to any known or unknown processes.

The terms “load-bearing magnetic eyeliner” or “magnetic eyeliner” of the present invention refer to eyeliner formulation incorporating one or more “magnetic particles” exhibiting strong ferromagnetic properties, and capable of supporting the physical attachment of magnetic eyelash prosthetic based on magnetic forces of attraction.

The term “magnetic particles” (incorporated into the magnetic eyeliner) refers to materials preferably selected from suitable magnetic compounds, including without limitation iron oxides, such as hematite Fe₂O₃, Magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂Fe³⁺₁₆O₂₇), Bismuth ferrite (BiFeO₃), Manganese-zinc ferrite (MnZn), Cobalt ferrite, CoFe₂O₄, Strontium ferrite, SrFe₁₂O₁₉, or yttrium iron granate (YIG) Y₃Fe₅O₁₂.

The term “magnetic eyelash prosthetic” of the present invention refers to the non-native eyelashes formed by incorporating “magnetic elements” into the “magnetic lash base,” which also functions by stably adhering a plurality of non-native eyelashes at their proximal end (opposite end to the eyelash tips). The term “magnetically attachable eyelash prosthetic” can be used interchangeably with “magnetic eyelash prosthetic.”

The term “magnetic lash base” refers to the structural component of the “magnetically attachable eyelash prosthetic” that can stably secure a plurality of eyelash hairs (non-native) at their proximal ends, and can incorporate “magnetic elements” positioned and oriented along the magnetic lash base, exhibiting high coercivity in the presence of the “load-bearing magnetic eyeliner” of the present invention.

The term “magnetic elements” (incorporated into the “magnetic lash base”) refers to permanent magnets of the hard or soft type, such as iron, cobalt, nickel; alloys, such as samarium-cobalt alloys, including SmCo₅, Sm₂Co17, optionally alloyed with Fe, Cu, and Zr; neodymium-iron-boron Nd₂Fe₁₄B optionally alloyed with Pr and Dy, commonly referenced as “rare-earth” or “super magnets”; and/or other alloys such as Bismanol and AlNiCo.

The term “magnetic” and “ferromagnetic” are used interchangeably in this disclosure without any limitations.

The terms “attachable” or “adherable” are used interchangeably in reference to the ability to physically interact with another surface of interest resulting in the bonding of two contacting surfaces, wherein the bonding is reversible as used in relation to the interaction between the “load-bearing magnetic eyeliner” coated over a skin of interest and the “magnetic eyelash prosthetic,” and in relation to the interaction between the “magnetic lash base” and the skin adhered/pre-coated “magnetic eyeliner.”

### B. The Magnetically Attachable Eyelash Prosthetic System Comprising a Magnetic Eyeliner

Consumers of eyelash-enhancing cosmetic products are very interested in using more advanced products having desirable properties that can be more suitable for their busy schedules and adaptations to various environmental circumstances. Women consumers especially appreciate beauty/cosmetic products that are intelligently designed, lighter in weight, healthier, aesthetically pleasing, comfortable and convenient to use (i.e., “user friendly” in meeting customer expectations). In particular, most women enjoy enhancing their appearance if the process/treatment is not challenging to implement. Many women choose not to fuss with cosmetic lashes and sticky glues because the application process is not very convenient, especially in waiting for the glue to dry and requiring manual dexterity, which may be wanting. The present inventor provides a convenient way for adhering eyelash prosthetics to consumers' eyelids without the mess or time wasted in fiddling with tacky/noxious glues, which may not be healthy when applied to delicate eyelids.

The inventive concept is directed to a magnetically attachable eyelash prosthetic system comprising two main components: a load-bearing magnetic eyeliner (“magnetic eyeliner”) and a magnetically attachable eyelash prosthetic (one or more) comprising a “magnetic lash base.” These two magnetic products are designed to magnetically attract each other during intended product use because the magnetic eyeliner incorporates “magnetic particles” suitable for cosmetics, wherein the “magnetic particles” can magnetically attract the “magnetic lash base” of the eyelash prosthetic, and thereby, supporting the stable attachment of the eyelash prosthetic.

In several embodiments, the present invention is directed to a load-bearing magnetic eyeliner capable of attaching one or more magnetic eyelash prosthetics, comprising: (a) a pigment suitable for eyelid cosmetic formulation; (b) one or more magnetic particles suitable for use in a pigmented cosmetic formulation; and (c) a load-bearing structural adhesive, capable of adhering to the surface of an eyelid and capable of magnetically supporting the attachment of a magnetic eyelash prosthetic, wherein the cosmetic formulation is convertible from a gel state to a load-bearing adhesive state after coating the skin surface; and wherein the adhesive force between the load-bearing structural adhesive of the skin-adhered eyeliner and the eyelid surface is greater than the magnetic force between the load-bearing eyeliner and the eyelash prosthetic, so that the eyelash prosthetic is reversibly attachable and detachable.

In several embodiments, the present invention is directed to a magnetically attachable eyelash prosthetic, comprising: (a) a lash base formed in part from a silicone polymer and incorporating one or more magnetic elements exhibiting high magnetic coercivity; and (b) a plurality of eyelash hairs adhered at the proximal end to the lash base, wherein the magnetic elements are positioned in a portion of the lash base, and arranged to face the surface of a magnetic eyeliner film formable over an eyelid.

In several embodiments, various methods for producing and using the various magnetically attachable eyelash prosthetics and the various load-bearing magnetic eyeliners are provided throughout the specification. Furthermore, examples 1-5 provides exemplary formulations for forming the load-bearing magnetic eyeliners, and Examples 6-10 provides exemplary formulations for forming the magnetically attachable eyelash prosthetics of the present invention.

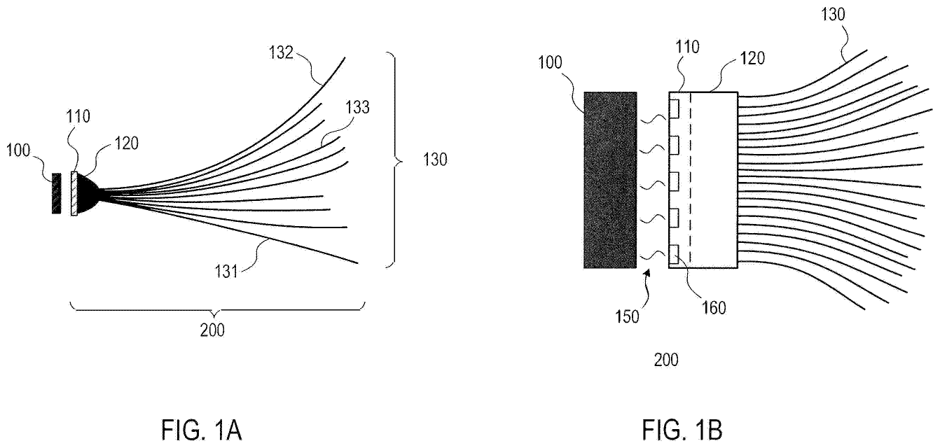

The magnetic interaction between the main components of the magnetically attachable eyelash prosthetic system can be explained using illustrations of FIGS. 1A and 1B described below.

FIG. 1A illustrates a simplified schematic of a magnetically attachable eyelash prosthetic system from a side perspective, showing the vertical special alignment between a load-bearing magnetic eyeliner and a magnetically attachable eyelash prosthetic, as one embodiment. In FIG. 1A, a magnetically attachable eyelash prosthetic 200 is shown in spatial vertical alignment relative to the load-bearing magnetic eyeliner 100 of the present invention (after pre-coating the eyelid surface). The magnetically attachable eyelash prosthetic 200 comprises a magnetic lash base 110 at the proximal end 120, wherein the individual lash hairs 131, 132, 133 (collectively, 130) can be stably secured. The magnetic lash base 110 exhibits magnetic properties by incorporating one or more “magnetic elements” (not shown in this figure but referenced in FIG. 1B). The magnetic lash base 110 can be attached securely to an eyelid surface prepared by pre-coating with the load-bearing magnetic eyeliner 100 of the present invention.

FIG. 1B illustrates a conceptual schematic of a magnetically attachable eyelash prosthetic system from a top perspective for facilitating the detailed descriptions herein, as one embodiment. In FIG. 1B, the magnetically attachable eyelash prosthetic system is shown as a conceptual model, wherein the magnetically attachable eyelash prosthetic 200 is positioned in the proximity of load-bearing magnetic eyeliner 100 in order to respond to the magnetic forces of attraction 150 across both interfacing surfaces. The eyelash prosthetic 200 comprises a magnetic lash base 110 at the proximal end 120 that stably secures a set of eyelash hairs 130 as shown. The magnetic lash base 110 can incorporate one or more magnetic elements 160, comprising various powderized/micronized ferromagnetic elements and ferromagnetic alloys or ferromagnetic compounds that can exhibit strong magnetic properties. The magnetic elements 160 can be structurally arranged in various ways, limited only by the human imagination (further detailed descriptions and examples are provided in other subsections).

### C. Methods for Stably Attaching and Reversibly Removing Eyelash Prosthetics by Incorporating Magnetic Elements

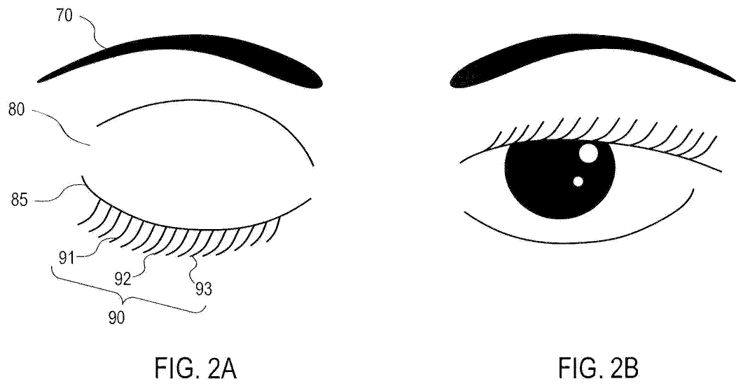

The process for attaching and detaching the magnetically attachable eyelash prosthetics of the present invention is illustrated and described in FIGS. 2-4 below.

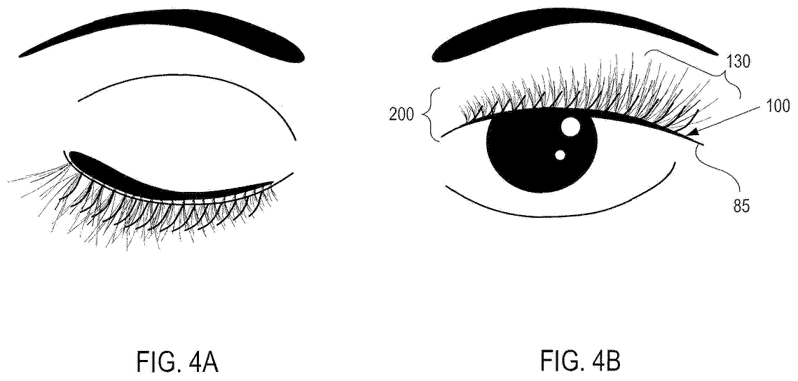

FIG. 2 A-B illustrate a hypothetical pair of eyes without any eyelash adornment, representing the condition prior to the attachment of magnetically attachable eyelash prosthetics of the present invention, as one embodiment. In FIG. 2A-B, the hypothetical pair of eyes without any eyelash adornment shows the sparsely distributed, native lashes, representing the pre-attachment stage before the application of the magnetically attachable eyelash prosthetic 200. FIG. 2A shows the eye-related features (in a closed-eye state), including the eyebrows 70, the eyelid 80, the contoured edge of the eyelid 85 representing a suitable surface for applying the load-bearing magnetic eyeliner 100 of the present invention, and some native lashes 91, 92, 93 (collectively, 90). FIG. 2B shows the same native features in an open-eye state, before adhering the magnetically attachable eyelash prosthetic 200 (as shown in FIG. 4 A-B).

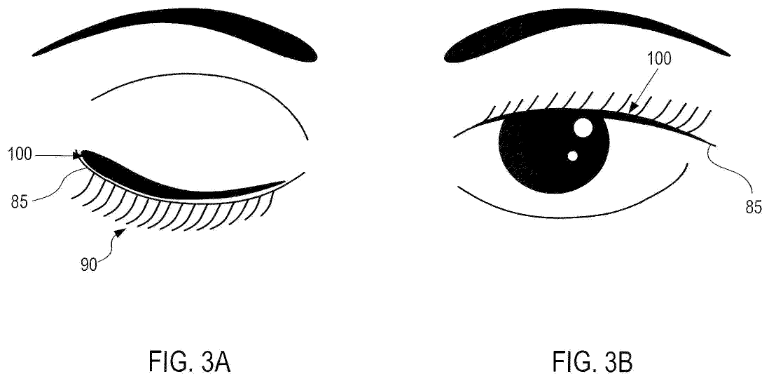

FIG. 3 A-B illustrate the application of the load-bearing magnetic eyeliner to the edge of the eyelid contour, as one embodiment. In FIG. 3 A-B, the application of the load-bearing magnetic eyeliner 100 to the edge of the eyelid contour 85 is shown. The load-bearing magnetic eyeliner 100 comprises a color pigment, one or more magnetic elements suitable for use in a pigmented cosmetic formulation, and a load-bearing structural adhesive. The load-bearing magnetic eyeliner 100 can be applied to the edge of the eyelid contour 85, utilizing a brush or a pointed applicator (not shown) of various shapes and sizes, and suitable for controlled application to achieve cosmetically pleasing results as shown. By pre-coating the load-bearing magnetic eyeliner 100 to the eyelid surface, the pre-coated eyeliner product placed on the eyelid surface can be converted from a liquid or semi-fluid state to a load-bearing adhesive state, so that the pre-coated eyeliner product can physically support magnetically attachable eyelash prosthetic 200 (as shown in FIG. 4 A-B) without peeling from the eyelid surface.

FIG. 4 A-B illustrate the stable attachment of the magnetically attachable eyelash prosthetic to the pre-coated, load-bearing magnetic eyeliner, as one embodiment. In FIG. 4 A-B, the magnetically attachable eyelash prosthetic 200 can be positioned into sufficient proximity to the load-bearing magnetic eyeliner 100 so that the magnetic eyelash prosthetic 200 attaches stably to the surface of the load-bearing magnetic eyeliner 100 (as a pre-coated form over an eyelid surface) as shown. The load-bearing magnetic eyeliner 100, comprising one or more “magnetic particles” suitable for use in a pigmented cosmetic formulation and a load-bearing structural adhesive, can be formulated to facilitate the magnetic attachment of the eyelash prosthetic 200 to achieve the desired outcome as shown in FIG. 4A (in a closed-eye state) and FIG. 4B (in an opened-eye state). The magnetically attachable eyelash prosthetic 200 can be reversibly attached/detached from the contoured edge of the eyelid 85 as shown, resulting in the extended/thicker lash appearance due to the adornment of a plurality of prosthetic lashes 130.

### D. Functional Components of Load-Bearing Magnetic Eyeliner Formulations

### Exemplary Structural Adhesives

In general, adhesives can be defined as substances capable of holding at least two surfaces together. A wide range of adhesive formulations are conceivable, and can be differentiated into either “pressure-sensitive adhesives” (PSAs) and “structural adhesives.” Pressure-sensitive adhesives (PSAs) can adhere strongly to solid surfaces upon application of light contact pressure and short contact duration, useful for manufacturing “post it” notes, adhesive pads, pressure-sensitive tapes, various labels, and a wide variety of products. Three different types of PSAs commercially employed are described as either: a) organic solvent based, b) water-based (emulsion), or c) thermoplastic. Pressure-sensitive adhesives are appreciated for their ease of application and removal from surfaces, but exhibit relatively less load-bearing capability than structural adhesives, and can be more prone to inadvertent detachment from substantially moist/oily surfaces, similar to most skin surfaces. Alternatively, structural adhesives can be defined as adhesives that “harden” or “cure” into a material capable of stably holding two or more substrates together during the expected lifetime of the product. Structural adhesives are often termed “load-bearing” adhesives, and exhibit higher load-bearing capabilities than aforementioned PSAs. Structural adhesives can be formulated utilizing various polymer chemistries, including polyurethane-, acrylic-, silicone-, vinyl-, or epoxy-based polymers and blends, among other suitable materials known by persons skilled in the art.

With respect to skin-contacting adhesive formulations, a variety of different types of adhesives can be utilized to enable skin-bonding. Suitable examples include various temporary dermal adhesives intended to support wound closure/wound repair. These formulations utilize among others, cyanoacrylate monomers and monomer blends formed from 2-octyl cyanoacrylate (CAS 133978-15-1) and/or n-Butylcyanoacrylate. Other dermal formulations may include Octyl 2-cyanoacrylate (CAS 6701-17-3), Isobutyl 2-cyanoacrylate and various materials that are functional equivalents.

Unfortunately, Cyanoacrylate formulations may be limited for use as a dermal adhesive in that they can be polymerized in the presence of water, and can polymerize rapidly (from tens of seconds to few minutes), adhering sufficiently well to the skin. These formulations must be stored dry to prevent their activation, since they can initiate curing upon contact with a moist skin. Thus, they must be applied comparatively quickly, and spread accurately. Because their adhesion to skin is quite substantial, and can last for a comparatively long time period (approximately one to several days), such formulations may not be suitable for formulating an eyeliner product in that: a) these formulations will not permit a sufficiently intermittent cosmetic application during 4-12 hr period, and b) the premature removal from a skin surface may require substantial force to be exerted and result in substantial discomfort. Cured formulations are also difficult to remove by traditional detergents, including soap and water, so that more volatile and potentially hazardous solvents, such as ethanol, ethyl acetate, or acetone may be needed to remove films/layers formed on the skin from such formulations. However, these types of solvents are known to degrease the skin and may cause skin irritations. Because the skin of the eye is very sensitive, it is desirable to avoid using such volatile, irritating and potentially hazardous solvents during eyeliner product use/product removal. Thus, there is a specific need to find more suitable skin-adhering formulations that do not detach as easily from the skin as aforementioned PSAs, and that does not adhere as strongly, as in the example of the aforementioned cyanoacrylate-based structural adhesives.

### Exemplary Pigments

The addition of pigments enables the eyeliner product to be formed in any desirable color from a spectrum of hues. Suitable pigments can be chosen from natural or synthetic, metallic, nonmetallic, inorganic pigments, organic pigments and/or pearlescent pigments, depending on the desired visual appearance, flowability, spreadability, dispersability, and/or wetting ability. Suitable pigments can be provided a) in various particle size ranges from typically tens of nanometers to about several hundred micrometers; b) with or without surface treatments; c) in hydrated or dehydrated form; and d) as is, or blended with each other, to reach a desired color spectrum. Exemplary inorganic pigments include black, yellow, red and brown iron oxides; titanium dioxide (rutile or anatase) (white), manganese violet; ultramarine blue; chromium oxide (green) and ferric blue, among others. Organic pigments include pigments D & C yellow, D & C orange, D & C red, carbon black and other dyes and/or lakes, among others. Pearlescent pigments include white pearlescent pigments, such as mica coated with titanium oxide or bismuth oxychloride; colored pearlescent pigments such as titanium mica with iron oxides, titanium mica with ferric blue or chromium oxide, titanium mica with an organic pigment of the abovementioned type, and pigments based on bismuth oxychloride, among others.

### Exemplary Magnetic Particles and Magnetic Elements

For producing the magnetically attachable eyelash prosthetic system, it is desirable to select (a) suitable “magnetic particles” exhibiting strong ferromagnetic properties for incorporation within the magnetic eyeliner formulations; and (b) suitable “magnetic elements” exhibiting strong ferromagnetic properties for incorporation within the “magnetic lash base.” These magnetic elements can be employed as: (i) a filler agent, as (ii) a color-imparting component, and (iii) a magnetic material that can facilitate the anchoring/attachment of the “magnetic lash base” to the present “load-bearing magnetic eyeliner.”

The ferromagnetic properties of the magnetic particles and the magnetic elements must be compatible in order for the magnetic eyelash prosthetic to attach stably to the magnetic eyeliner (eyelid skin-adhered/pre-coated). As a preferred embodiment, a ferromagnetic material exhibiting high coercivity is suitable for use in forming “magnetic particles” incorporated within the “load-bearing magnetic eyeliner” formulation, preferably in the range between 10-25,000 kA/m, more preferably from 100-15,000 kA/m and most preferably from 1,000-10,000 kA/m. As a preferred embodiment, a “magnetic element,” having an intrinsically high coercivity is incorporated into the “magnetic lash base,” preferably in the range between 10-25,000 kA/m, more preferably from 100-15,000 kA/m and most preferably from 1,000-10,000 kA/m.

As another embodiment, suitable materials for forming the “magnetic elements” for incorporation into the “magnetic lash base” include powderized/micronized ferromagnetic elements, ferromagnetic alloys and ferromagnetic compounds, typically used in permanent magnets of the hard or soft type, such as iron, cobalt, nickel; alloys, such as samarium-cobalt alloys, including SmCo₅, Sm₂Co17, optionally alloyed with Fe, Cu, and Zr; neodymium-iron-boron Nd₂Fe₁₄B optionally alloyed with Pr and Dy, commonly referenced as “rare-earth” or “super magnets”; and/or other alloys such as Bismanol and AlNiCo.

As another embodiment, suitable materials for forming “magnetic particles” for incorporation into the “magnetic eyeliner” include ferromagnetic compounds such as oxide-based magnetic pigments, including without limitation iron oxides, such as hematite Fe₂O₃, Magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂Fe³⁺₁₆O₂₇), Bismuth ferrite (BiFeO₃), Manganese-zinc ferrite (MnZn), Cobalt ferrite, CoFe₂O₄, Strontium ferrite, SrFe₁₂O₁₉, or yttrium iron granate (YIG) Y₃Fe₅O₁₂.

As another embodiment, the “magnetic elements” and “magnetic particles” can be provided in encapsulated or unencapsulated form to optimize properties such as suspendability, dispersability, surface wetting characteristics, dermal compatibility, and resistance from environmental factors, including oxidation resistance. For example, iron oxides can be prevented from environmental degradation or direct skin contact by the addition of masking agents, including fatty acids, such as undecylenic, lauric or oleylic acid.

With regard to suitable “magnetic particle” sizes for use in the magnetic eyeliner formulation, generally, the preferred size ranges cannot easily be visually distinguished from one another by the naked eye, thus avoiding a granular appearance. As several embodiments, the magnetic particles exhibit an average diameter size ranging from 1 μm to 1 mm, preferably ranging from 5 μm to 500 μm, and most preferably ranging from 50 μm to 250 μm. As another embodiment, the magnetic particles exhibit an average diameter size ranging from 100 nm to 1 μm, wherein the magnetic particles can be further encapsulated to inhibit sub-dermal transport.

Black iron oxides, which are available in various size ranges and chemistries, are deemed particularly useful, because they impart not only a rich black color, but also confer the desired magnetic properties to the underlying formulation. A suitable overall composition range for such iron oxide based magnetic particles can range from between 10-60 (w/w %), more preferably 20-50 (w/w %), and most preferably from 25-45 (w/w %). It should be noted, that the higher the coercivity of the “magnetic particle” chosen as the pigment component, the lesser amount need be incorporated to achieve a desired magnetic adhesion force for stably attaching a “magnetic lash base” to a complementarily formulated “magnetic eyeliner.” An analogous rationale can be applied with respect to the incorporation of magnetic elements into the “magnetic lash base.” Thus, the needed magnetic adherent force can be finely controlled through material selection and the relative amount of “magnetic elements” added to the “magnetic lash base” with respect to the amount of “magnetic particles” added to the “magnetic eyeliner.”

### Suitable Adhesive and Viscosifying Components

The inventors of the present application have found that suitable skin-adhering formulations can be formed on the basis of acrylate polymer blends, preferably from methyl and ethyl acrylate, 2-hydroxyethylacrylate, butyl acrylate, isobutyl acrylate, t-butyl methacrylate, isooctyl acrylate, ethyl hexyl acrylate, isobornyl acrylate, vinyl acetate, and more particular from: ethyl hexyl acrylate copolymers in an overall weight ratio of 0-30 (w/w %), more preferably 5-25 (w/w %) and most preferably from 10-15 (w/w %) in combination with butyl acrylate, methyl methacrylate, and methacrylic acid copolymers, in an overall weight ratio of 5-45 (w/w %), more preferably 10-30 (w/w %) and most preferably from 15-25 (w/w %), that can be selected from a molecular weight range of 5,000-30,000 g/mol, more preferably from 10,000-20,000 g/mol and most preferably from 15,000-20,000 g/mol. The latter polymer blends can be particularly suitable for forming a structural adhesive capable of stably adhering to the eyelid skin.

### Suitable Thickening Agents

Depending on the desired flowability and spreadability for an optimized application to the skin, viscosifying-, gelling-, thickening-, bulking- and/or thixotropic agents can be added to facilitate the blending with other acylates in the liquid formulations. Examples include gelling agents based on natural gums, including carob gum, guar gum, gum arabic, karaya gum, gum tragacanth and ghatti gum, agar-agar, carrageenans, alginates, gelatin, caseinates, albumins, pectins, starches, polysaccharides, such as xanthan gum, chitin and/or chitosanes, polydextranes such as carboxymethyl dextranes, cellulose and cellulose derivatives, such as carboxymethylcellulose, hydroxypropylcellulose, methylcellulose, hydroxyl-propylmethylcellulose or hydroxyethylcellulose, and cellulose derivatives modified by alkyl- or alkoxy-groups. Others include inorganic filler materials, and/or pigments, such as silicate minerals, talcs or hydrated magnesium silicates, micas or aluminosilicates, such as for example muscovite, margarite, roscoelite, lipidolite, biotite, sericite, hectorite and/or bentonite, kaolin or hydrated aluminium silicate, boron nitride, fumed silica and/or titanium dioxide.

Naturally occurring resins, fragrances, oils, and waxes of plant or animal derived materials among others can be added to further enhance the performance of the eyeliner.

### Suitable Film-Forming Components

In formulating the load-bearing magnetic eyeliner, other desirable properties include cosmetic functions such as film-forming and opacifying-controlling properties that can facilitate the formation of a homogenous, visually pleasing opaque film surface. The inventor of the present application noted that these properties can be enhanced through the addition of styrene/methacrylate/acrylate copolymer blends, including, but not limited to methacrylic acid-styrene copolymer and/or their respective alkali salts. Suitable overall weight ratios in a formulation comprising such styrene/acrylate copolymer blends can include 5-45 (w/w %), more preferably 10-30 (w/w %) and most preferably from 15-25 (w/w %). In the event, that more water resistant film-forming properties are desirable, fluorinated acrylate polymers, such as 2,2,2-Trifluoroethyl methacrylate, 2,2,3,3-Tetrafluoropropyl methacrylate, 2,2,3,4,4,4-Hexafluorobutyl methacrylate, 2,2,3,3,3-Pentafluoropropyl acrylate, and/or 1,1,1,3,3,3-Hexafluoroisopropyl acrylate can be added to the blend in a suitable proportion to weatherproof the surface.

### Suitable Water Content

Overall water weight ratios for the provided formulations can be chosen from between 0-50 (w/w %) more preferably from 0-25 (w/w %) and most preferably from 5-15 (w/w %). Upon application, the water evaporates up to a residual content (e.g. equivalent to the moisture retained in the skin), substantially solidifying the adhesive formulation in the process, and thereby creating a stable structural support and adherence on the skin substrate.

### Desired Viscosity Ranges

The viscosity can be adjusted through a desired amount of viscosifying agents, thickening agents, and/or filling agents, particularly including various pigments. The viscosity of the formulations in the underlying disclosure typically can range from 5 cps-300 cps, which can be measured between about the viscosity of water and castor oil (or higher viscosity).

The aforementioned polymer blends do not cure in the presence of water because they are not provided as a monomer, and are already cured. They can be readily dissolved in water, forming viscous to gel-like solutions and are suitably provided in a non-crosslinked state. Because the formulation is formed partially from water-soluble, non-crosslinked polymers, the magnetic eyeliner film/layer can be easily removed from the skin by employing conventional detergent solutions and associated skin-care products intended for make-up removal.

### Emollients/Humectants

To further control the residual moisture content and hardness of the adherent layer, various humectifying-, emollifying-, softening- and/or anti-caking agents can be added to the eyeliner formulation. Suitable humectants can include ethylene-, propylene-, butylene- and hexylene glycols, as well as their multifunctional alcohol derivatives, alpha-hydroxy acids such as lactic acid, glyceryl triacetate, polymeric polyols such as polydextrose, sodium hexametaphosphate, sugar alcohols such as glycerol, sorbitol, xylitol, maltitol, and urea. Suitable emollients can include such as castor oil, cetyl alcohol, cetearyl alcohol, cocoa butter, isopropyl myristate, isopropyl palmitate, lanolin, liquid paraffin, polyethylene glycols, shea butter, silicone oils, stearic acid, and stearyl alcohol. Suitable humectifying-, emollifying-, softening- and/or anti-caking agents concentrations in the overall composition can range from between 0.01-20 (w/w %), more preferably 0.1-15 (w/w %), and most preferably from 1-10 (w/w %).

### Surfactants and Dispersants

To achieve a homogeneous dispersion of the substantially solid components of the formulation, the addition of surfactants and emulsifying agents such as saturated and unsaturated polyoxyethylene (2-80) alkyl (C8-20) ethers, for example Polysorbate 20-80, or polyethylene glycol ethers derived from castor oil, for example Ricinoleth-40 can be added.

### Preservatives

Other suitable components include preservatives with antifungal and antimicrobial activity, including, but not limited to benzoic acid, propionic acid, and their salts, esters of p-hydroxybenzoic acid, aromatic alcohols such as benzyl alcohol or phenoxyethanol, unsaturated and/or saturated fatty acids, such as undecylenic acid, lauric acid, oleic acid and/or stearic acid, sodium dehydroacetate, and other structural or functional equivalents, to prevent microbial growth and spoilage of the formulation during storage. The overall composition range for the specified surfactants and preservatives can typically range from about 0-10 (w/w %), more preferably 0.1-5 (w/w %), and most preferably from about 0.5-3 (w/w %).

Several exemplary formulations for making the load-bearing magnetic eyeliner are further described in Examples 1-5. Alternative substitutions that are conceivable by persons skilled in the art of cosmetic formulations are contemplated within the scope of the present invention.

### E. Various Structural Configurations for Forming the Magnetic Lash Base for the Eyelash Prosthetic

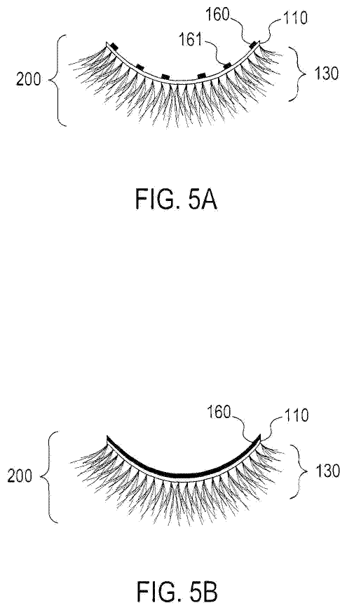

FIGS. 5A-B provides several embodiments to show that the magnetic elements can be incorporated, internally or externally, with respect to the “magnetic lash base” that physically secures the lash hairs at their proximal ends. There are many variations that are contemplated, and these figures represent only a few design configurations.

FIG. 5A illustrates the magnetically attachable eyelash prosthetic showing physically attachable magnetic elements, as one embodiment. In FIG. 5A, the magnetically attachable eyelash prosthetic 200 is shown, wherein the magnetic elements 160, 161 can be physically attached to the exterior surface of the magnetic lash base 110, which can stabilize/affix a plurality of lashes 130 at the proximal end.

FIG. 5B illustrates the magnetically attachable eyelash prosthetic incorporating magnetic elements into the lash base, as another embodiment. In FIG. 5B, the magnetically attachable eyelash prosthetic 200 is shown, wherein the magnetic elements 160 can be physically incorporated within the magnetic lash base 110 to produce a uniform or homogeneous external surface (seamless or minimal surface irregularities) that contacts the skin. The magnetic lash base 110 also functions by stabilizing/affixing a plurality of lashes 130 at the proximal end as shown.

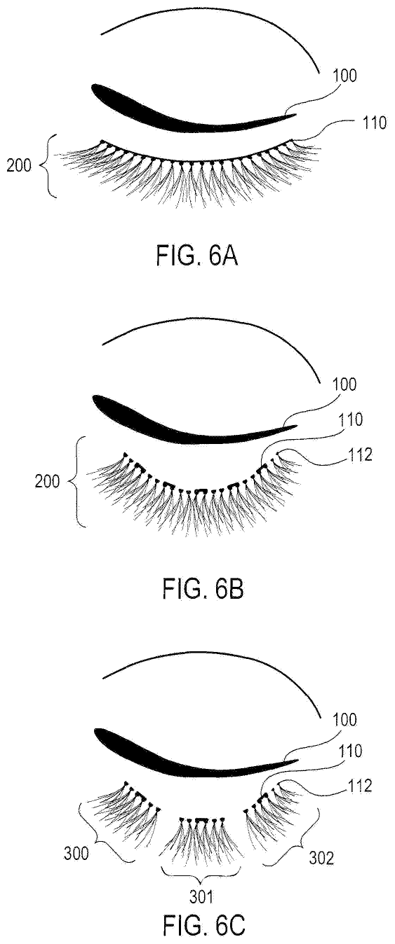

FIG. 6A illustrates the magnetically attachable eyelash prosthetic comprising a magnetic lash base formed as a single continuous unit capable of magnetically attaching to a load-bearing magnetic eyeliner, as another embodiment. In FIG. 6A, the magnetically attachable eyelash prosthetic 200 is shown, wherein a plurality of lash hairs can be stably attached to a magnetic lash base 110 formed as a single continuous unit and reversibly attachable/detachable to the magnetic eyeliner 100 of the present invention. This configuration can provide full-width coverage, or may be reduced in size by the consumers to suit their particular cosmetic needs using for example, a pair of shears. All color, texture, thickness, and length variations of the lash hairs are contemplated.

FIG. 6B illustrates the magnetically attachable eyelash prosthetic comprising a magnetic lash base formed as a dis-continuous unit, as another embodiment. In FIG. 6B, the magnetically attachable eyelash prosthetic 200 is formed, wherein multiple smaller fragments of lashes can be grouped together and stably attached to a shorter magnetic lash base fragment 110, 112 as shown. This configuration can provide substantial flexibility to the magnetic lash base to suit the particular cosmetic needs of each user. All color, texture, thickness, and length variations of the lash hairs are contemplated.

FIG. 6C illustrates the magnetically attachable eyelash prosthetic formed by attaching several shorter prosthetic units arranged in tandem, as another embodiment. In FIG. 6C, the magnetically attachable eyelash prosthetic is formed, wherein several shorter prosthetic units 300, 301, 302 (representing shorter segments of a full-width lash prosthetic) can be arranged to span a range from the inner corner to the outer corner of an eye as shown. Each shorter prosthetic unit can be directly attached to a different, non-overlapping region along the eyelid contour after pre-coating the surface with the load-bearing magnetic eyeliner 100 of the present invention. The shorter segments of the magnetic base 110, 112 incorporating one or more suitable magnetic elements are referenced herein. This configuration can provide substantial design options for each user by providing the opportunity to custom select different color and/or lash weight (texture) selections for each sub-region of their eyelid/eye for adornment (i.e., inner corner, middle, and outer edge). All color, texture, thickness, and length variations of the lash hairs are contemplated.

### F. Exemplary Formulations/Processes for Producing Magnetically Attachable Eyelash Prosthetics

The magnetically attachable eyelash prosthetic comprises: (a) a lash base incorporating one or more magnetic elements exhibiting high magnetic coercivity; and (b) a plurality of eyelash hairs adhered at the proximal end to the lash base, wherein the magnetic elements are positioned towards the surface of the lash base capable of contacting and attaching the surface of a magnetic eyeliner film formed over an eyelid. The “magnetic lash base,” “magnetic elements,” and “eyelash hairs” are further described below.

The “magnetic lash base” can be manufactured from a wide range of polymeric materials, provided that these materials can be formed substantially flexible and conforming to the skin, have adequate dermal compatibility without significant irritation potential, and are easily blended with the contemplated magnetic elements. Suitable polymers capable of forming an eyelash base include fluoroelastomers, polysulfones, polyamides, polyurethanes, polyesters, polyethers, silicones, polycarbonates, polyurethane carbonates, polyesters, polyamides, polyimides, polyvinyls, and polyolefins, polyvinyl alcohols, polyacetates, including blends and mixtures thereof.

Particularly suited for a skin-contacting application can be polyurethane and silicone based materials, due to their intrinsically low irritation potential, good dermal compatibility and a widely adjustable range of flexibility. These polymer materials are available with a wide range of elastic properties, for example, that can be expressed through a measurement of shore hardness A, which can range from 0 to about 90 durometers. For the given application, ranges smaller than 50 durometers, more preferably smaller than 40 durometers and most preferably smaller than 30 durometers can be considered an optimum range of softness. In addition, the aforementioned polymers possess excellent compounding properties, and can be processed through a wide range of methods ideally suitable for blending with contemplated “magnetic elements.”

The magnetic elements are not limited as to the exact disposition of the polymer substrate utilized for forming the contemplated “magnetic lash base.” For example, the magnetic elements can be incorporated, adhered, layered, reacted, blended/mixed, embedded, grafted, bonded, compounded, crosslinked, copolymerized and/or reacted with the monomers for forming the polymer substrate, or can be processed as part of an intermediate layer that can be adhered, adjoined, affixed and/or reacted, or combined with the polymer substrate utilized for forming the “magnetic lash base” in any manner. Furthermore, the magnetic elements can be combined with a conventional polymer, and the combination can be adhered onto/around/within the “magnetic lash base” or the surface of the device so that the magnetic element and the polymer substrate can be deposited simultaneously or sequentially. The magnetic elements can be incorporated into the polymer substrate of the “magnetic lash base” through various processes, including dispersion, suspension, sedimentation, encapsulation, coating, layering, film deposition, sputtering, spraying and similar functional equivalent. Depending on the particular process applied, the spatial distribution of the magnetic elements dispersed in the “magnetic lash base” can be substantially isotropic, or anisotropic, homogeneous, or inhomogeneous, or in the form of a gradient or non-gradient distribution.

Because the magnetic adherent force between the surface of a first ferromagnetic material (e.g., magnetic element) and the surface of a second ferromagnetic material (e.g., magnetic particles) (towards each other) depends on the orientation and distance between them, the smaller the gap existing between the “magnetic elements” incorporated into the “magnetic lash base” and the “magnetic particles” in the “magnetic eyeliner,” the stronger the resulting adherent force. Thus, the magnetic elements in the “magnetic lash base” are preferably arranged in close local proximity to the surface of the eyeliner. To achieve this specific spatial arrangement, the magnetic particles can be sedimented by gravitation within the monomer solution during the curing process of the polymer substrate utilized for forming the “magnetic lash base.” Alternatively, a magnetic force can be utilized to apply reverse gravitational pull on the magnetic elements when suspended in the monomer solution, such that the particles are preferably oriented not at the bottom, but at the top of the substrate.

The foregoing description, for purposes of explanation, refers to specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously, many modifications and variations are possible in view of the above teachings. The embodiments are shown and described in order to best explain the principles of the invention and practical applications, to thereby enable others skilled in the art to best utilize the invention and various embodiments with various modifications as suitable for the particular uses contemplated. It is intended that the scope of the invention be defined by the following claims and their equivalents.

### EXAMPLES

### Example 1

### Exemplary Structural Adhesive Formulation for the Load-bearing Magnetic Eyeliner Employing Iron (II,III) Oxide Nanocrystals (SPIO)

As several embodiments, exemplary structural adhesive formulations capable of stably adhering to the eyelid skin can include the following agents:

| | | Composition: | | |
|---|---|---|---|---|
| | | A | B | C |
| Function | Ingredient(s) | (w/w) % | (w/w) % | (w/w) % |
| Magnetic Element(s) | Iron (II, III) oxide nanocrystals (SPIO) | 35.0 | 40.0 | 45.0 |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum/ | 1.0 | 1.0 | 1.0 |
| | Cellulose/ | | | |
| | Clay Mineral | | | |
| Emollient/ | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat/ | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt/ | | | |
| | (2E,4E)-hexa-2,4-dienoic acid/ | | | |
| | 2-phenoxyethanol/ | | | |
| | Undec-10-enoic acid | | | |
| Solvent(s) | Water | 10.0 | 10.0 | 10.0 |

### Example 2

### Exemplary Structural Adhesive Formulation for the Load-bearing Magnetic Eyeliner Employing Iron (II,III) Oxide Nanopowder (>50-100 nm Particle Size)

As several embodiments, exemplary structural adhesive formulations capable of stably adhering to the eyelid skin can include the following agents:

| | | Composition: | | |
|---|---|---|---|---|
| | | D | E | FC |
| Function | Ingredient(s) | (w/w) % | (w/w) % | (w/w) % |
| Magnetic Element(s) | Iron (II, III) oxide nanopowder, >50-100 | 35.0 | 40.0 | 45.0 |
| | nm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum/ | 1.0 | 1.0 | 1.0 |
| | Cellulose/ | | | |
| | Clay Mineral | | | |
| Emollient/ | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat/ | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt/ | | | |
| | (2E,4E)-hexa-2,4-dienoic acid/ | | | |
| | 2- phenoxyethanol/ | | | |
| | Undec-10-enoic acid | | | |
| Solvent(s) | Water | 10.0 | 10.0 | 10.0 |

### Example 3

### Exemplary Structural Adhesive Formulation for the Load-Bearing Magnetic Eyeliner Employing Iron (II,III) Oxide Micronized Powder (<5 μm Particle Size)

As several embodiments, exemplary structural adhesive formulations capable of stably adhering to the eyelid skin can include the following agents:

| | | Composition: | | |
|---|---|---|---|---|
| | | G | H | I |
| Function | Ingredient(s) | (w/w) % | (w/w) % | (w/w) % |
| Magnetic Element(s) | Iron (II, III) oxide micronized | 35.0 | 40.0 | 45.0 |
| | powder, <5 μm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum/ | 1.0 | 1.0 | 1.0 |
| | Cellulose/ | | | |
| | Clay Mineral | | | |
| Emollient/ | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat/ | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt/ | | | |
| | (2E,4E)-hexa-2,4-dienoic acid/ | | | |
| | 2-phenoxyethanol/ | | | |
| | Undec-10-enoic acid | | | |
| Solvent(s) | Water | 10.0 | 10.0 | 10.0 |

### Example 4

### Exemplary Structural Adhesive Formulation for the Load-Bearing Magnetic Eyeliner Neodymium-Iron-Boron Nd₂Fe₁₄B Powder (>50-60 μm Particle Size)

As several embodiments, exemplary structural adhesive formulations capable of stably adhering to the eyelid skin can include the following agents:

| | | Composition: | | |
|---|---|---|---|---|
| | | J | K | L |
| Function | Ingredient(s) | (w/w) % | (w/w) % | (w/w) % |
| Magnetic Element(s) | Neodymium-iron-boron Nd2Fe14B | 35.0 | 40.0 | 45.0 |
| | powder, >50-60 μm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum/ | 1.0 | 1.0 | 1.0 |
| | Cellulose/ | | | |
| | Clay Mineral | | | |
| Emollient/ | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat/ | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt/ | | | |
| | (2E,4E)-hexa-2,4-dienoic acid/ | | | |
| | 2-phenoxyethanol/ | | | |
| | Undec-10-enoic acid | | | |
| Solvent(s) | Water | 10.0 | 10.0 | 10.0 |

### Example 5

### Exemplary Structural Adhesive Formulation for the Load-bearing Magnetic Eyeliner Employing Barium Ferrite BaFe₁₂O₁₉ Powder (>40-50 μm Particle Size)

As several embodiments, exemplary structural adhesive formulations capable of stably adhering to the eyelid skin can include the following agents:

| | | Composition: | | |
|---|---|---|---|---|
| | | J | K | L |
| Function | Ingredient(s) | (w/w) % | (w/w) % | (w/w) % |
| Magnetic Element(s) | Barium ferrite BaFe12O19 | 35.0 | 40.0 | 45.0 |
| | powder, >40-50 μm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum/ | 1.0 | 1.0 | 1.0 |
| | Cellulose/ | | | |
| | Clay Mineral | | | |
| Emollient/ | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat/ | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt/ | | | |
| | (2E,4E)-hexa-2,4-dienoic acid/ | | | |
| | 2- phenoxyethanol/ | | | |
| | Undec-10-enoic acid | | | |
| Solvent(s) | Water | 10.0 | 10.0 | 10.0 |

### Example 6

### Exemplary Structural Configuration for the Magnetic Lash Base Formed as a Single Silicone Layer

For creating a suitable “magnetic lash base” comprising magnetic elements, the following process can be applied to form it as a single silicone layer. A silicone RTV or platinum cure compound can be mixed with the desired quantity of magnetic elements in a non-polar solvent such as heptane. A vacuum is applied to a silicone solution in order to remove the solvent and residual trapped gases. The silicone solution can be poured onto a first plate, containing a plurality of “eyelash base-shaped” depressions, or molds. The amount of solution is adjusted such that a desired film thickness can be reached. Sufficient time is allowed for the magnetic elements to settle to the bottom of the mold. Alternatively, a second, magnetized plate can be positioned over the first plate, so that the magnetic elements can settle at the top of the mold. The silicone compound can be left to substantially cure as needed. The set silicone film can be removed from the mold. The desired “magnetic lash base” external contours can be cut out using mechanical, pneumatic and/or laser cutting methods. The proximal ends of eyelash hairs can be adequately positioned at each “magnetic lash base” contour. A silicone adhesive compound is utilized to fixate the oriented eyelashes onto the pre-formed “magnetic lash base” and left to cure. The resulting “magnetic lash base” can be formed as a single silicone layer with a gradient arrangement of dispersed magnetic elements.

As one particular example, 100.0 g of Iron (II,III) oxide micronized powder, <5 μm particle size is added to a 100.0 g of a 0.001 M solution of Undec-10-enoic acid in n-heptane. The resulting slurry can be stirred for a period of 20 minutes at room temperature, and poured into a 1.0 L volume of a Part B (base) of platinum cure type Nusil MED-6600 silicone dispersion. The mixture can be homogeneously blended under the application of a vacuum until n-heptane and residual gases have been substantially removed. Subsequently, a 1.0 L volume of Part A (accelerator) of platinum cure type Nusil MED-6600 silicone dispersion can be added to the prepared iron-oxide suspension dispersed in silicone base B. The silicone Part A:B mixture can be homogeneously blended under the application of a vacuum until residual gases have been substantially removed, or for a period of about 5-10 min. The homogeneously dispersed 2.02 L silicone Iron (II,III) oxide suspension can be poured onto a rectangular mold plate of dimensions 200 cm×100 cm and left to cure at 25° C. for a period of at least 45 minutes, yielding a cured silicone layer height of 1 mm and having an Iron (II,III) oxide area density of about 0.005 g/cm², wherein through means of sedimentation, the particles can be located facing the bottom of the mold plate. After the desired “magnetic lash base” external contours have been cut out from the above 1 mm silicone layer, each lash base is contacted (on the opposite side facing the magnetic elements) with about 100 μL of Nusil MED-1137 RTV silicone adhesive, followed by securing the pre-arranged lashes into place.

As one preferred embodiment, the base silicone layer without magnetic elements has a Shore A hardness of about 25 durometers, that will be respectively higher by the proportion of magnetic elements added. When lower ultimate shore hardness is desired, silicone oil-based thinning agents, such as Dow Corning 360 medical fluid 1000 cSt, or any other suitable polydimethylsiloxane oil, can be added to the pre-cure mixture, such that the shore hardness of the cured silicone layer can be adjusted in a range of about 0-30 durometers.

Depending on the desired area density of magnetic elements distributed in the silicone layer intended for manufacturing the eyelash base, the amount of magnetic elements can be easily adjusted (by providing a respectively higher amount thereof in the formulation provided above). In the provided example, an initial quantity of 200.0 g of Iron (II,III) oxide can yield an area density of about 0.01 g/cm², 500.0 g of about 0.025 g/cm², and 1000.0 g of about 0.05 g/cm². Higher amounts are contemplatable, when required for the application.

In another embodiment, the magnetic elements of Example 6 can be replaced with the magnetic elements named in examples 1, 2, 4 and 5. Therefore, by choice of the desired magnetic element, as well as the amount added to, and spatial location within the eyelash base, suitable magnetic properties can be conveyed onto the eyelash base, thereby yielding a magnetic eyelash base with adequate load-bearing capability, when placed onto a load-bearing magnetic eyeliner.

The amounts and area densities of magnetic elements to be formulated in the eyelash base can be obtained through practical quantitative experimentation. For example, a metered amount of magnetic eyeliner can be spread over a defined surface area on a test substrate reflective of the eyelid surface. The test substrate is turned upside down, and the magnetic eyelash base is magnetically attached to the magnetic eyeliner. A base acceptance criterion is reached when the magnetic force between the “magnetic eyelash base” and the “magnetic eyeliner” exceeds the gravitational pull on the “eyelash base,” and when the adhesive force between the eyeliner and the test substrate exceeds the gravitational pull exerted on the magnetic eyelash base.

### Example 7

### Exemplary Structural Configuration for the Magnetic Lash Base Formed as a Dual Silicone Layer

For creating a suitable “magnetic lash base” comprising magnetic elements, the following process can be applied to form it as a dual silicone layer. A pre-cured silicone sheet can be placed on a plate. A silicone adhesive formulation containing a desired quantity of magnetic elements dispersed within a non-polar solvent such as hexane can be sprayed onto the pre-cured sheet. The topcoat can be left to dry as needed. The “magnetic lash base” contours can be cut out using mechanical, pneumatic and/or laser cutting methods. The proximal ends of eyelash hairs can be adequately positioned at each “magnetic lash base” contour. A silicone adhesive compound can be utilized to fixate the oriented eyelashes onto the pre-formed “magnetic lash base” and left to cure. The resulting “magnetic lash base” can be formed as a dual silicone layer configuration comprising a top silicone layer of dispersed magnetic elements positioned over a base silicone layer.

### Example 8

### Exemplary Structural Configuration for the Magnetic Lash Base Formed as a Single Silicone Layer (Wire-Shaped Magnetic Elements)

For creating a suitable “magnetic lash base” comprising magnetic elements, the following process can be applied to form it as a single silicone layer. The “magnetic lash base” contours can be obtained from a pre-cured silicone sheet. A magnetic element in a wire form can be threaded through each base. The proximal ends of eyelash hairs can be adequately positioned at each “magnetic lash base” contour. A silicone adhesive compound can be utilized to fixate the oriented eyelashes onto the pre-formed “magnetic lash base” and left to cure. The resulting “magnetic lash base” can be formed as a dual silicone layer configuration comprising a single silicone layer configuration further comprising a wire-shaped magnetic element embedded therein.

### Example 9

### Exemplary Structural Configuration for the Magnetic Lash Base Formed as a Dual Silicone Layer (Any-Shape Magnetic Elements)

For creating a suitable “magnetic lash base” comprising magnetic elements, the following process can be applied to form it as a dual silicone layer. A plurality of spherical, cube or otherwise shaped magnetic elements can be positioned on top of a pre-cured silicone sheet. A silicone adhesive formulation can be sprayed onto the pre-cured sheet and left to cure. The “magnetic lash base” contours comprising the pre-positioned magnetic elements are cut out using mechanical, pneumatic and/or laser cutting methods. The proximal ends of eyelash hairs can be adequately positioned at each “magnetic lash base” contour. A silicone adhesive compound can be utilized to fixate the oriented eyelashes onto the pre-formed “magnetic lash base” and left to cure. The resulting “magnetic lash base” can be formed as a dual layer silicone configuration comprising magnetic elements formed as spherical, cube or any shape of interest embedded therein.

### Example 10

### Exemplary Structural Configuration for the Magnetic Lash Base Formed as a Triple Polymer Layer

For creating a suitable “magnetic lash base” comprising magnetic elements, the following process can be applied to form it as a single silicone layer. A thin film polymer substrate comprising pre-positioned magnetic elements can be co-extruded together with a top and bottom layers of the same or different polymers. The resulting “magnetic lash base” can be formed as a triple polymer layer configuration comprising “pre-positioned magnetic elements” embedded therein.

1-30. (canceled)

31. A method comprising:
applying an eyeliner in a liquid or gel state onto an eyelid surface, the eyeliner comprising an acrylate polymer and further comprising ferromagnetic particles;
adhering the eyeliner to the eyelid surface by permitting the eyeliner to maintain contact with the eyelid surface for an amount of time sufficient to convert the eyeliner from the liquid or gel state to a hardened load-bearing state that forms an adhesive force between the acrylate polymer and the eyelid surface; and
forming a stable attachment between (i) the eyeliner in the hardened load-bearing state adhered to the eyelid surface and (ii) a base of an eyelash prosthetic, the eyelash prosthetic further comprising a plurality of eyelash hairs extending from the base, the eyelash prosthetic comprises one or more ferromagnetic elements positioned at the base, the forming of the stable attachment comprises positioning the base of the eyelash prosthetic in proximity to the eyeliner sufficient for magnetic force between the ferromagnetic particles of the eyeliner and the one or more ferromagnetic elements of the eyelash prosthetic;
wherein the adhesive force between the eyelid surface and the acrylate polymer of the eyeliner adhered to the eyelid surface exceeds the magnetic force between the ferromagnetic particles of the eyeliner and the one or more ferromagnetic elements of the eyelash prosthetic, and wherein the magnetic force between the ferromagnetic particles of the eyeliner and the one or more ferromagnetic elements of the eyelash prosthetic exceeds gravitational force on the eyelash prosthetic, and thereby, the magnetic force between the ferromagnetic particles of the eyeliner and the one or more ferromagnetic elements of the eyelash prosthetic stably attaches the eyelash prosthetic to the eyelid surface.

32. The method of claim 31, wherein the acrylate polymer in the eyeliner is an acrylate polymer blend.

33. The method of claim 32, wherein the acrylate polymer blend has a molecular weight range of 5,000-30,000 g/mol, preferably from 10,000-20,000 g/mol, and more preferably from 15,000-20,000 g/mol.

34. The method of claim 32, wherein the acrylate polymer blend comprises one or more of methyl acrylate, ethyl acrylate, 2-hydroxyethylacrylate, butyl acrylate, isobutyl acrylate, t-butylmethacrylate, isooctyl acrylate, ethyl hexyl acrylate, isobornylacrylate or vinyl acetate.

35. The method of claim 32, wherein the acrylate polymer blend comprises ethyl hexyl acrylate copolymers in combination with one or more of a butyl acrylate copolymer, a methyl methacrylate copolymer, or a methacrylic acid copolymer.

36. The method of claim 31, wherein the ferromagnetic particles of the eyeliner have a coercivity from 10 kA/m to 25,000 kA/m, preferably from 100 kA/m to 15,000 kA/m, and most preferably from 1,000 kA/m to 10,000 kA/m.

37. The method of claim 31, wherein the ferromagnetic particles of the eyeliner are selected from the group consisting of iron oxides; hematite Fe₂O₃; magnetite Fe^{II}(Fe^{III})₂O₄; super-paramagnetic iron oxides (SPIO); chromium dioxide CrO₂; barium ferrites that are one or more of BaFe₁₂O₁₉, Ba₂ZnFe₁₈O₂₃ or BaFe²⁺2Fe³⁺₁₆O₂₇; bismuth ferrite BiFeO₃; manganese-zinc ferrite MnZn; cobalt ferrite CoFe₂O₄; strontium ferrite SrFe₁₂O₁₉; yttrium iron granate Y₃Fe₅O₁₂; and mixtures thereof.

38. The method of claim 31, wherein the ferromagnetic particles of the eyeliner comprise black iron oxide.

39. The method of claim 38, wherein the black iron oxide is 10-60 w/w % of the eyeliner, more preferably 20-50 w/w % of the eyeliner, and most preferably from 25-45 w/w % of the eyeliner.

40. The method of claim 38, wherein the eyeliner further comprises at least one fatty acid in an amount effective to mitigate or prevent at least one of environmental degradation of the black iron oxide or direct contact of the black iron oxide with the eyelid surface, the at least one fatty acid selected from the group consisting of undecylenic acid, lauric acid, and oleylic acid.

41. The method of claim 31, wherein the ferromagnetic particles of the eyeliner have an average diameter size from 1 μm to 1 mm, preferably from 5 μm to 500 μm, and most preferably from 50 μm to 250 μm.

42. The method of claim 31, wherein the eyeliner further comprises a film-forming component.

43. The method of claim 42, wherein the film-forming component comprises a methacrylic acid-styrene copolymer.

44. The method of claim 43, wherein the methacrylic acid-styrene copolymer is 5-45 w/w % of the eyeliner, preferably 10-30 w/w % of the eyeliner, and more preferably from 15-25 w/w % of the eyeliner.

45. The method of claim 31, wherein the acrylate polymer of the eyeliner comprises a fluorinated acrylate polymer.

46. The method of claim 45, wherein the fluorinated acrylate polymer is selected from the group consisting of 2,2,2-trifluoroethyl methacrylate; 2,2,3,3-tetrafluoropropyl methacrylate; 2,2,3,4,4,4-hexafluorobutyl methacrylate; 2,2,3,3,3-pentafluoropropyl acrylate; 1,1,1,3,3,3-hexafluoroisopropyl acrylate; and mixtures thereof.

47. The method of claim 31, wherein water is between 0 and 50 w/w % of the eyeliner, preferably between 0 and 25 w/w % of the eyeliner, and more preferably from 5 to 15 w/w % of the eyeliner.

48. The method of claim 31, wherein the eyeliner in the liquid or gel state has a viscosity from 5 cps to 300 cps.

49. The method of claim 31, wherein the eyeliner further comprises at least one agent selected from the group consisting of ethylene glycol, propylene glycol, butylene glycol, hexylene glycol, alpha-hydroxy acids, glyceryl triacetate, polymeric polyols, sodium hexametaphosphate, sugar alcohols, castor oil, cetyl alcohol, cetearyl alcohol, cocoa butter, isopropyl myristate, isopropyl palmitate, lanolin, liquid paraffin, polyethylene glycols, shea butter, silicone oils, stearic acid, and stearyl alcohol.

50. The method of claim 49, wherein the at least one agent is 0.01-20 w/w % of the eyeliner, preferably 0.1-15 w/w % of the eyeliner, and more preferably from 1-10 w/w % of the eyeliner.

51. The method of claim 31, wherein the eyeliner further comprises a surfactant.

52. The method of claim 51, wherein the surfactant is selected from the group consisting of saturated and unsaturated polyoxyethylene alkyl ethers.

53. The method of claim 31, wherein the base of the eyelash prosthetic comprises a polymer comprising at least one of polyurethane or silicone.

54. The method of claim 53, wherein the polymer has shore hardness A below 90 durometers, preferably smaller than 50 durometers, more preferably smaller than 40 durometers, and most preferably smaller than 30 durometers.

55. The method of claim 31, wherein the one or more ferromagnetic elements of the eyelash prosthetic have a coercivity from 10 kA/m to 25,000 kA/m, preferably from 100 kA/m to 15,000 kA/m, and most preferably from 1,000 kA/m to 10,000 kA/m.

56. The method of claim 31, wherein the one or more ferromagnetic elements of the magnetic eyelash prosthetic are selected from the group consisting of bismanol, AlNiCo. neodymium-iron-boron Nd₂Fe₁₄B, and mixtures thereof.

57. The method of claim 31, wherein the eyelash prosthetic has at least one configuration selected from the group consisting of (a) at least a portion of the one or more ferromagnetic magnetic elements are physically attached to an exterior surface of the base of the eyelash prosthetic and (b) at least a portion of the one or more ferromagnetic elements are physically incorporated within the base of the eyelash prosthetic such that the eyelash prosthetic has an external surface that is at least one of uniform or homogeneous.

58. The method of claim 31, wherein the eyelash prosthetic has a configuration selected from the group consisting of (a) the base is a single continuous unit attached to the plurality of eyelash hairs and (b) the base comprises separate sections, each of the sections attached to a corresponding portion of the plurality of eyelash hairs.

59. The method of claim 31, further comprising removing the eyelash prosthetic from the eyeliner after the stable attachment of the eyelash prosthetic to the eyeliner, with the eyeliner still adhered to the eyelid surface; and then reforming the stable attachment of the eyelash prosthetic to the eyeliner adhered to the eyelid surface.

50. The method of claim 31, wherein the stable attachment of the eyelash prosthetic to the eyelid surface consists of (a) the adhesive force between the eyelid surface and the acrylate polymer of the eyeliner adhered to the eyelid surface and (b) the magnetic force between the ferromagnetic particles of the eyeliner and the one or more ferromagnetic elements of the eyelash prosthetic.
